(19) United States
(12) Patent Application Publication
(10) Pub. No.: US 2023/0346956 A1
(43) Pub. Date: Nov. 2, 2023

(54) COMPLEX OF 7-DEACETYLFORSKOLIN AND PVP

(71) Applicant: SciPharm S.à.r.l, Mertert (LU)

(72) Inventor: Andreas KUBIN, Eichgraben (AT)

(21) Appl. No.: 17/794,946

(22) PCT Filed: Jan. 22, 2021
(86) PCT No.: PCT/EP2021/051499

§ 371 Date: Jul. 22, 2022

(30) Foreign Application Priority Data
Jan. 23, 2020 (EP) 20153429.4

(51) Int. Cl. A61K 47/58

(52) U.S. Cl. CPC A61K 47/58

(57) ABSTRACT

An object of the invention is a complex of 7-deacetyl-forskolin (7-DAFSK) and polyvinylpyrrolidone (PVP). Furthermore, the invention relates to a process for preparing such a 7-DAFSK-PVP complex and to a pharmaceutical composition containing such a complex.

### FIELD OF THE INVENTION

The present invention relates to a complex of 7-deacetyl-forskolin (7-DAFSK) and polyvinylpyrrolidone (PVP) and a method for preparing such a complex.

### BACKGROUND OF THE INVENTION

The diterpene forskolin is a virtually water-insoluble plant compound from Plectranthus barbatus (Coleus forskohlii). Forskolin is in focus for versatile medical applications, including cardiovascular disease, bronchial asthma, obesity, treatment of glaucoma, pulmonary disease, joint inflammation, and osteoarthritis/arthritis. Forskolin promotes cartilage formation in joints and at the same time prevents ossification and mineralization. Furthermore, forskolin is being studied in stem cell research with regard to differentiation and regression to pluripotency.

Forskolin activates adenylyl cyclase, a membrane-bound enzyme. Adenylyl cyclase converts adenosine triphosphate (ATP) to cyclic adenosine monophosphate (cAMP) and releases cAMP into the cytosol, increasing the intracellular concentration of cAMP in most cells and tissues (Metzger H. et al., 1981, Drug Research, 1248-50). cAMP acts as a second messenger in cellular signal transduction and serves to activate many peptide hormones (protein kinases).

Forskolin is a diterpene with two α-hydroxy groups at position 1 and 9, a β-hydroxy group at position 6, and a β-acetoxy group at position 7. The two α-hydroxy groups at position 1 and 9 are essential for binding to adenylyl cyclase. Forskolin, due to its low solubility in water (0.01 mg/mL, WO2005025500A2), needs different formulations to be used as a diagnostic or therapeutic agent. Such formulations contain various solvents such as alcohols or DMSO, which are often not well tolerated and lead to side effects.

Document U.S. Pat. No. 6,346,273B1 discusses a method for increasing the water solubility of poorly water-soluble pharmaceutically active substances, such as forskolin, by complexation with a non-ionic polymer, such as polyvinylpyrrolidone (PVP). This document describes an increase in the water solubility of forskolin.

Increased water solubility of forskolin is achieved by complexing forskolin with cyclodextrins. WO2005025500A2 describes a method for complexing forskolin with cyclodextrin in an aqueous medium.

WO2017103840A1 describes water-soluble forskolin compositions by complexation with water-soluble polymers such as PVP, among others. After mixing forskolin with the polymer, a starch solution is added and the mixture is heated, then cooled and spray-dried.

WO2018127600A1 discloses a method for preparing forskolin-cyclodextrin complexes, which is based on a sintering process and allows an increased content of forskolin in the complex, as well as increased water solubility.

Another approach to increase the water solubility of forskolin is chemical derivatization. A forskolin derivative with higher water solubility compared to forskolin is 7-deacetyl forskolin, which shows a similar effect on adenylyl cyclase as forskolin (Laurenza A. et al, Molecular Pharmacology 1987, 32(1), 133-139, Pinto C. et al, Biochemical Pharmacology 2009, 78(1), 62-69). However, deacetylation of forskolin at position 7 can only increase water solubility to a limited extent (up to 1 mg/mL).

Therefore, there is a need for an improved water-soluble form of a forskolin derivative, which is characterized by both high water solubility and thus improved bioavailability, and high forskolin derivative content.

It is therefore an object of the present invention to provide a form of 7-deacetyl-forskolin having increased solubility in water and, at the same time, a high content of 7-deacetyl-forskolin.

### SUMMARY OF THE INVENTION

The object is solved by a complex of 7-deacetyl-forskolin (7-DAFSK) and polyvinylpyrrolidone (PVP), as described in the claims and embodiments of the present invention.

The present invention discloses a complex of 7-deacetyl-forskolin (7-DAFSK) and polyvinylpyrrolidone (PVP).

In one embodiment, the complex according to the invention is characterized in that the average mass fraction of 7-DAFSK in the total complex is in the range of 1 to 50 wt %, preferably 5, 10, 15, 20 or 25 wt %, more preferably 30 wt %, 35 wt %, 40 wt %, 45 wt %, or higher.

In one embodiment, the average molar ratio of 7-DAFSK to PVP in the complex is 0.1 or higher, preferably 1, 2, 3, 4, 5, 6, 7, 8, 9, 10, 20, 30, 40, 50, 60 or higher, more preferably 3 or higher.

In one embodiment, the complex according to the invention is characterized in that PVP has an average molar mass of from 1 to 40 kD. Preferably, the PVP has an average molar mass of 1 to 25 kD, more preferably about 2 to 3 kD, most preferably about 2.5 kD.

In a further embodiment, the complex according to the invention is characterized in that 7-DAFSK is derived from synthetic forskolin.

In one embodiment, the 7-DAFSK is present in a purity of at least 98.5%.

It was surprisingly found that a particularly high mass fraction of 7-DAFSK in the 7-DAFSK-PVP complex can be achieved by heating a mixture of 7-DAFSK and PVP to a temperature that is above the glass transition temperature of the PVP used. In particular, the mixture is heated to a temperature that is at least 20° C., preferably at least 25° C., 30° C., 40° C., 50° C., 60° C., 70° C., 80° C., 90° C., 100° C. above the glass transition temperature of the PVP. In particular, the temperature is brought to a range of about 25 to 50° C. above the glass transition temperature of the PVP.

Therefore, the present invention also provides a method for preparing the 7-DAFSK-PVP complex according to the invention, which is characterized in that a mixture of 7-DAFSK and PVP is heated to a temperature higher than the glass transition temperature of the PVP used.

In one embodiment, the process according to the invention is characterized in that a solvent, preferably water, ethanol, methanol, acetone, ethyl methyl ketone, dichloromethane and/or ethyl acetate, even more preferably water, ethanol, methanol, propanol and/or dichloromethane, is added to the mixture of 7-DAFSK and PVP. Particularly preferably, water and/or ethanol, or a mixture thereof, is added to the mixture of 7-DAFSK and PVP.

In a particular embodiment, the mixture according to the invention is kept at a temperature higher than the glass transition temperature of the PVP used for at least 5 minutes.

In one embodiment, the method according to the invention includes the following steps:
a) Preparing a mixture of 7-DAFSK and PVP and adding a solvent, preferably an alcohol, preferably ethanol, to the mixture,
b) Addition of water,
c) Heating the mixture, above the glass transition temperature of the PVP for at least 5 minutes,
d) Cooling of the mixture and dissolving in water,
e) Removal of the undissolved components, optionally by filtering.
   In one particular application form, the mixture is heated at least 20, 25, 30, 35, 40, 45, 50, 55, 60° C., 70° C., 80° C., 90° C., 100° C. above the glass transition temperature of the PVP.

The application of the method according to the invention allows the efficient and straightforward preparation of 7-DAFSK-PVP complexes, which significantly increase the water solubility of 7-DAFSK and may also contain a high mass fraction of 7-DAFSK, enabling beneficial applications of 7-DAFSK in diagnosis and/or therapy.

With the complexes of 7-DAFSK with PVP according to the invention, the water solubility of 7-DAFSK can be increased. By using a high mass fraction of 7-DAFSK in the complex according to the invention, the water solubility of 7-DAFSK can be increased without thus requiring an unfavorably large amount of PVP. Formulations with forskolin derivatives can thus also be assembled in a more material-saving manner than before. By avoiding large amounts of PVP and associated side effects, the present invention facilitates and improves the use of 7-DAFSK in diagnosis and/or therapy.

Therefore, the present invention also provides a pharmaceutical composition containing a 7-DAFSK-PVP complex according to the invention.

In one embodiment, said pharmaceutical composition is characterized in that the composition comprises 7-DAFSK at a concentration of at least 25 mg/L, 50 mg/L, 75 mg/L, 100 mg/L, 200 mg/L, 300 mg/L, 400 mg/L, 500 mg/L, 600 mg/L, 800 mg/L, 1000 mg/L, 2000 mg/L, 3000 mg/L, or 4000 mg/L, preferably 4000 mg/L.

One aspect of the present invention relates to a pharmaceutical composition for oral, intravenous or inhalation administration.

In a particular embodiment, the pharmaceutical composition according to the invention is used in a therapeutic method.

In another particular embodiment, the pharmaceutical composition according to the invention is used for the treatment of cardiovascular diseases, bronchial asthma, obesity, glaucoma, pulmonary diseases, joint inflammation, or osteoarthritis/arthritis.

One aspect of the present invention relates to a pharmaceutical composition according to the invention for use as a medicament.

### FIGURES

FIG. 1: Reaction scheme for the deacetylation of forskolin to 7-DAFSK by sodium carbonate (Na₂CO₃).

FIG. 2: Comparison of the mass fraction of 7-DAFSK on 7-DAFSK-PVP K12 and 7-DAFSK-PVP K25 complexes in samples prepared by the heating method and the solution method.

FIG. 3: Proportion of converted 7-DAFSK at different weight ratios of DAFSK to PVP K12 used.

FIG. 4: Comparison of the mass fraction of 7-DAFSK on 7-DAFSK-PVP K12 complexes at different weight ratios of 7-DAFSK to PVP K12.

FIG. 5: Relationship between water solubility and the mass fraction of 7-DAFSK in the 7-DAFSK-PVP K12 complex.

FIG. 6: cAMP assay using HEK293 cells containing EP2, EP4 and IP receptors. Cells were stimulated with a control sample (treprostinil), 30 μM forskolin in DMSO, 100 μM 7-DAFSK in water and 100 μM 7-DAFSK in 7-DAFSK-PVP K12 complex in water.

### DETAILED DESCRIPTION

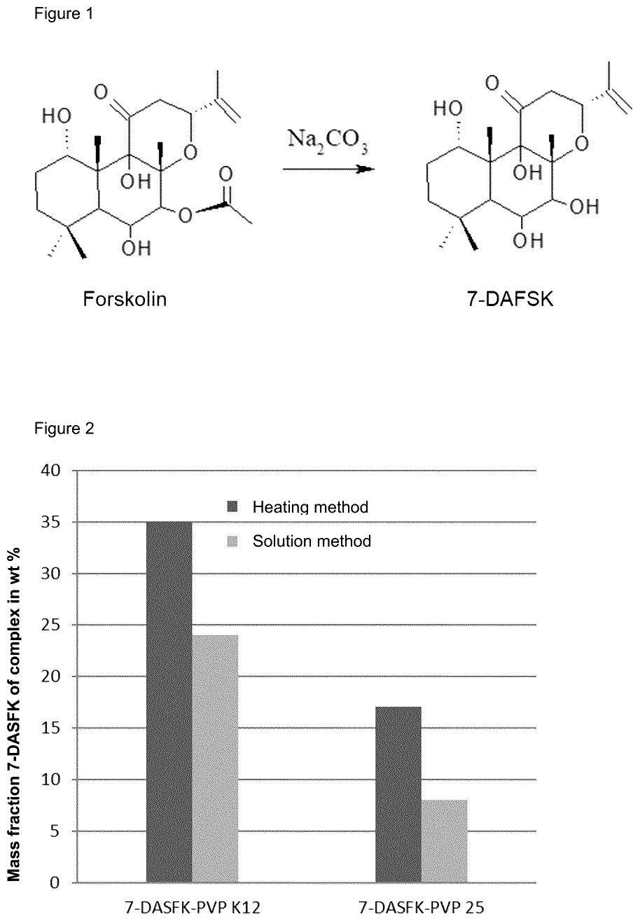

7-deacetyl-forskolin (7-DAFSK) can be represented by the structural formula shown in FIG. 1. 7-DAFSK may also exist in other forms besides the molecular form, such as in the form of salts, for example, alkali metal salts such as sodium or potassium salts. In the context of the present invention, the term “7-deacetyl-forskolin (7-DAFSK)” refers to both the molecular form, as shown in the structural formula in FIG. 1, and all other possible forms, such as salts of 7-DAFSK, preferably alkali metal salts, particularly preferably a sodium or potassium salt.

Polyvinylpyrrolidone (PVP), also polyvidone or povidone, is a polymer of the compound vinylpyrrolidone. PVP is commercially available in different degrees of polymerization. The degree of polymerization determines the average molar mass of the polymer. The term “average molar mass” as used herein refers to the mass average of the molar mass.

In the context of the present invention, PVP with an average molar mass of 1 to 40 kD is preferred because polymers of this size can still be excreted well and unmetabolized by the kidney.

In one embodiment, the 7-DAFSK-PVP complex is characterized in that the PVP has an average molar mass of from 1 to 60 kD, in particular from 1 to 40 kD. Preferably, PVP has an average molar mass of from 1 to 25 kD, preferably from about 2 to 9 kD, from 2 to 8 kD, from 2 to 7 kD, from 2 to 6 kD, from 2 to 5 kD, from 2 to 4 kD, more preferably from 2 to 3 kD, most preferably from about 2.5 kD.

The term “about” herein refers to the specified value, or a value that deviates from the specified value by +/−10%.

The molecular weight of PVP can also be defined by the K-value, which is obtained by viscosity measurement and depends on the viscosity average of the molar mass, instead of specifying the mass average of the molar mass. PVP with a mass average molecular weight of about 2.5 kD corresponds to PVP with a K-value of about 12, referred to here as “PVP K12” (K. Kolter et al. “Hot-Meld Extrusion with BASF Pharma Polymers,” ISBN 978-3-00-039415-7, 2012). PVP with a weight average molecular weight of about 24 kD corresponds to PVP with a K-value of about 25, referred to here as “PVP K25”.

In the context of the present invention, the term “7-DAFSK-PVP complex” refers to a chemical product containing 7-DAFSK and PVP and characterized by the fact that a linkage exists between 7-DAFSK and PVP. The term “complex” does not limit the nature of the compound in any way, but simply means that there is a linkage between one or more 7-DAFSK molecules and one or more PVP molecules. The linkage may be, for example, a covalent bond or, preferably, a non-covalent attachment of 7-DAFSK to PVP, e.g., through van der Waals bonds or hydrogen bonds. The term “7-DAFSK-PVP” or “7-DAFSK-PVP complex” should be understood to comprise the chemical product as a whole and not a single 7-DAFSK-PVP complex at the molecular level.

The terms “mass fraction” and “molar ratio” are always to be understood herein as average values. They do not refer to each individual complex at the molecular level, but to an average value of the entire chemical product.

Percentages (%) in the present invention refer in each case to percent by weight (wt %), unless otherwise indicated.

In one embodiment, the average mass fraction of 7-DAFSK in the total complex is 1 to 50 wt %, preferably 2, 3, 4, 5, 6, 7, 8, 9, 10, 15, 20 or 25 wt %, more preferably 30 wt %, 35 wt %, 40 wt %, 45 wt % or higher. The mass fraction here reflects the relative proportion of the mass of 7-DAFSK to the mass of the total complex (7-DAFSK-PVP).

The proportion of 7-DAFSK in the 7-DAFSK-PVP complex can also be expressed as a molar ratio. For example, a 7-DAFSK-PVP complex consisting of molecular 7-DAFSK (molar mass: 368.5 g/mol) and PVP having an average molar mass of about 2.5 kD (“PVP K12”), and having an average mass fraction of 7-DAFSK in the total complex of 30 wt %, an average molar ratio of 7-DAFSK to PVP in the complex of about 3. The average molar ratio here reflects the ratio of the molar amount of 7-DAFSK to the molar amount of PVP.

In one embodiment of the present invention, the average molar ratio of 7-DAFSK to PVP in the complex is 0.1 or higher, preferably 3, 4, 5, 6, 7, 8, 9, 10, 15, 20, 25, 30, 35, 40, 45, 50, 55, 60 or higher, more preferably 3 or higher.

The proportion (mass fraction as well as molar ratio) of 7-DAFSK in the total complex is always to be understood herein as the average proportion. A suitable method for determining the average proportion of 7-DAFSK in the total complex is high-performance liquid chromatography (HPLC). The skilled person is familiar with the performance of such a determination.

7-DAFSK can be prepared from forskolin by means of a deacetylation reaction, e.g., carbonate saponification, as explained in the specific examples of the present invention. Both natural and synthetic forskolin can be used as the reactant. Natural forskolin is typically present as an enriched extract, which is purified prior to use. In the context of the present invention, synthetic forskolin, which is commercially available in a high purity, is preferably used as the reactant for the 7-DAFSK synthesis.

In one embodiment, the complex according to the invention is therefore characterized in that 7-DAFSK is derived from synthetic forskolin. In a preferred embodiment, the 7-DAFSK is present in a purity of at least 98.5%.

The 7-DAFSK-PVP complex according to the invention is characterized by increased water solubility compared to non-complexed 7-DAFSK-PVP, which is advantageous for pharmaceutical applications.

Surprisingly, it was found that 7-DAFSK-PVP complexes are easily accessible synthetically by heating 7-DAFSK and PVP to a temperature higher than the glass transition temperature of the PVP used. Surprisingly, this procedure also leads to a high mass fraction of 7-DAFSK in the overall complex.

The present invention therefore comprises a process for preparing a 7-DAFSK-PVP complex according to the invention, characterized in that a mixture of 7-DAFSK and PVP is heated to a temperature higher than the glass transition temperature of the PVP used.

As an amorphous substance, PVP has no melting point, but exhibits a so-called glass transition temperature. The glass transition temperature depends, among other things, on the degree of polymerization, i.e. the average molar mass, of PVP (see Table 1).

TABLE 1
Examples of the glass transition temperature
of PVP as a function of molar mass.

| | Glass transition |
|---|---|
| Average molar mass of PVP | temperature |
| approx. 2.5 kD (,,PVP K12“) | 93° C. |
| approx. 9 kD (,,PVP K17“) | 130° C. |
| approx. 24 kD (,,PVP K25“) | 155° C. |
| approx. 40 kD (,,PVP K30“) | 175° C. |

In this context, the “glass transition temperature of the PVP used” should be understood as the temperature at which the glass transition of PVP in the present mixture with 7-DAFSK occurs. The glass transition temperature of the PVP used can be influenced by the composition of the mixture, for example if solvents or water are added to the mixture. Methods for determining the glass transition temperature are known to those skilled in the art. Preferably, the glass transition temperature can be determined following the procedure of the corresponding DIN standard (Wampfler B., et al., Messunsicherheit in der Kunststoffanalytik—Ermittlung mit Ringversuchsdaten. Carl Hanser Verlag, Munich 2017, ISBN-10: 3446452869).

A particularly preferred embodiment of the process according to the invention for the preparation of the 7-DAFSK-PVP complex is characterized in that the average molar mass of the PVP used is about 2.5 kD (“PVP K12”), and the mixture is heated to a temperature above 93° C. Specifically, heating is performed to a temperature that is at least 25° C., particularly 30° C., 40° C., 45° C. or more above the glass transition temperature of PVP K12.

Another preferred embodiment is characterized in that the average molar mass of the PVP used is about 9 kD (“PVP K17”) and the mixture is heated to a temperature above 130° C. More specifically, it is heated to a temperature that is at least 25° C., particularly 30° C., 40° C., 45° C. or more above the glass transition temperature of PVP K17.

Another preferred embodiment is characterized in that the average molar mass of the PVP used is about 24 kD (“PVP K25”) and the mixture is heated to a temperature above 155° C. More specifically, it is heated to a temperature that is at least 25° C., in particular 30° C., 40° C., 45° C. or more above the glass transition temperature of the PVP K 25.

Another preferred embodiment is characterized in that the average molar mass of the PVP used is about 40 kD (“PVP K 30”) and the mixture is heated to a temperature above 175° C. More specifically, it is heated to a temperature that is at least 25° C., in particular 30° C., 40° C., 45° C. or more above the glass transition temperature of the PVP K 30.

It has been found advantageous to add a solvent or a mixture of solvents to the mixture of 7-DAFSK and PVP according to the invention. Preferably, the mixture of 7-DAFSK and PVP is stirred paste-like in little solvent. The addition of solvent can help to homogeneously distribute 7-DAFSK and PVP and allow larger amounts of 7-DAFSK to adhere to PVP. Suitable solvents are aqueous, alcoholic or other organic solvents, such as water, ethanol, methanol, propanol, acetone, ethyl methyl ketone, dichloromethane and/or ethyl acetate.

In one embodiment, the process according to the invention for the preparation of the 7-DAFSK-PVP complex is characterized in that a solvent, preferably water, ethanol, methanol, propanol, acetone, ethyl methyl ketone, dichloromethane and/or ethyl acetate, even more preferably water, ethanol, methanol, propanol, dichloromethane and/or pyridine, is added to the mixture of 7-DAFSK and PVP. Particularly preferably, water and/or ethanol, or a mixture thereof, is added to the mixture of 7-DAFSK and PVP. The addition of water and/or ethanol may be simultaneous or sequential.

It has been found useful in the manufacturing process to keep the mixture of 7-DAFSK and PVP at a temperature higher than the glass transition temperature of the PVP used for a certain period of time. Good results were obtained when the mixture was kept above the glass transition temperature for at least 5 minutes.

In a particular embodiment of the process according to the invention, the mixture of 7-DAFSK and PVP is therefore kept at a temperature higher than the glass transition temperature of the used PVP for at least 5 minutes.

In a particular embodiment, the method according to the invention comprises the following steps, which are carried out in sequence:
a) Preparing a mixture of 7-DAFSK and PVP and adding a solvent, preferably an alcohol, preferably ethanol, to the mixture,
b) Addition of water,
c) Heating the mixture above the glass transition temperature of the PVP for at least 5 minutes,
d) Cooling of the mixture and dissolving in water,
e) Removal of the undissolved components, optionally by filtering.

In one embodiment, a mixture of 7-DAFSK and PVP is prepared in a weight ratio of 2:1 to 1:4, preferably 2:1, 2:1.5, 1:1, 2:3, 1:2, 1:3, and 1:4, most preferably 1:1 or 1:2.

The application of the method according to the invention allows the efficient and straightforward preparation of 7-DAFSK-PVP complexes.

The process according to the invention allows the preparation of 7-DAFSK-PVP complexes with a high mass fraction of 7-DAFSK. Conventional methods for preparing PVP complexes involve dissolving the components in a solvent or solvent mixture, where the mixture is not heated above the glass transition temperature of PVP. In a specific example explained herein, the process of the invention achieved 8-9% higher average mass fractions of 7-DAFSK in 7-DAFSK-PVP complexes compared to conventional complexation processes.

A high mass fraction of 7-DAFSK in 7-DAFSK-PVP complexes is particularly advantageous for pharmaceutical applications in diagnosis and/or therapy. By using a high mass fraction of 7-DAFSK in the complex according to the invention, the water solubility of 7-DAFSK can be increased without requiring an unfavorably large amount of PVP. Formulations with forskolin derivatives can thus also be assembled in a more material-saving manner than before. By avoiding large amounts of adjuvants (PVP) and associated side effects, the present invention facilitates and improves the use of 7-DAFSK in diagnosis and/or therapy.

The 7-DAFSK-PVP complex according to the invention can be used for any dietary or medical purpose in which forskolin and/or 7-DAFSK is applicable.

Therefore, the present invention also relates to a pharmaceutical composition containing the 7-DAFSK-PVP complex of the invention with 7-DAFSK as the active ingredient.

The term “pharmaceutical composition” is used herein in reference to the 7-DAFSK-PVP complex according to the invention with 7-DAFSK as active ingredient, optionally in combination with further ingredients and/or active ingredients.

The pharmaceutical composition according to the invention may be present in a variety of systemic and topical formulations. Non-limiting examples of systemic or topical formulations containing the 7-DAFSK-PVP complex of the invention include oral, intrabuccal, intrapulmonary, rectal, intrauterine, intradermal, topical, dermal, parenteral, intratumoral, intracranial, buccal, sublingual, nasal, subcutaneous, intravascular, intrathecal, inhalable, respirable, intraarticular, intracavitary, implantable, transdermal, ionotophoretic, intraocular, vaginal, optical, intravenous, intramuscular, intraglandular, intraorganic, intralymphatic formulations, enteric coatings, or slow-release formulations.

The composition may be administered once or several times daily. A preferred embodiment relates to a pharmaceutical composition according to the invention for oral, intravenous or inhalation administration.

Formulations for oral administration may be provided in discrete units such as capsules, tablets, or lozenges containing the formulation as powder or granules. Formulations for oral administration may also be provided as a solution or suspension in aqueous or non-aqueous medium, or as an emulsion.

Formulations for intravenous administration can be provided as a solution or suspension in an aqueous medium, e.g., as a saline solution.

The specific dosage of 7-DAFSK-PVP complex to achieve a therapeutic or prophylactic effect depends on the specific application, e.g., the route of administration, the age, weight, and condition of the individual patient, the disease being treated, and the severity of symptoms.

The dose of 7-DAFSK-PVP in the composition according to the invention is preferably an effective dose that achieves a therapeutic or prophylactic effect without causing undesirable side effects, and the composition can be administered as a single dose or in multiple units.

In a particular embodiment, the composition according to the invention is provided as a solution or aerosol comprising 7-DAFSK at a concentration of at least 25 mg/L, preferably at least 50 mg/L, 75 mg/L, 100 mg/L, 200 mg/L, 300 mg/L, 400 mg/L, 500 mg/L, 600 mg/L, 800 mg/L, or 1000 mg/L. In a preferred embodiment, the composition contains 7-DAFSK at a concentration of at least 1 mg/mL, 1.5 mg/mL, 2 mg/mL, 2.5 mg/mL, 3 mg/mL, or 4 mg/mL, most preferably 4 mg/mL.

In another particular embodiment, the composition according to the invention is provided as a capsule or tablet containing 7-DAFSK in a pharmaceutically effective amount, for example 10 mg to 2000 mg, preferably at least 25 mg, 50 mg, 100 mg, 200 mg, 300 mg, 400 mg, 500 mg, 600 mg, 700 mg, 800 mg, 900 mg or 1000 mg, more preferably 500 mg.

In a particular embodiment, the pharmaceutical composition according to the invention is used in a therapeutic method.

Forskolin and 7-DAFSK are known to be non-specific cAMP stimulators. The 7-DAFSK-PVP complex according to the invention can be used to treat any disease or disorder that can be treated with forskolin and/or 7-DAFSK. Non-limiting examples of such diseases include neurodegenerative diseases, Alzheimer's disease, motor dysfunction, acute and chronic cardiovascular diseases, pulmonary diseases such as asthma, cystic fibrosis, vascular diseases associated with cystic fibrosis, bronchitis, chronic obstructive pulmonary disease (COPD), obesity, glaucoma, joint inflammation, osteoarthritis/arthritis, fibrotic changes such as idiopathic pulmonary fibrosis, post-traumatic pulmonary fibrosis, bronchopulmonary dysplasia (BPD), post-toxic liver disease such as VPD or cirrhosis, and peripheral circulation disorders including Raynaud's disease and scleroderma.

In a particular embodiment, the pharmaceutical composition according to the invention is used for the treatment of cardiovascular diseases, bronchial asthma, obesity, glaucoma, pulmonary diseases, joint inflammation, or osteoarthritis/arthritis.

Another aspect of the present invention relates to a pharmaceutical composition according to the invention for use as a medicament.

The present invention is further illustrated by, but not limited to, the following examples and figures.

### EXAMPLES

The following examples describe the synthesis of 7-DAFSK and the complexation of 7-DAFSK with, where PVP with an average molar mass of about 2.5 kD (“PVP K12”) and about 24 kD (“PVP K25”) was chosen, and the amount ratios of 7-DAFSK to PVP were varied. The examples further address the characterization of the obtained complexes in terms of the content of 7-DAFSK, the water solubility, and the effect as a cAMP activator in cAMP assays. The examples do not include detailed descriptions of standard methods, such as performing a content determination by HPLC, or measuring NMR spectra. Such methods are well known to the person skilled in the art.

### Example 1—Synthesis of 7-deacetyl Forskolin

Mercachem's synthetic forskolin (GMP—material), batch MAMA07-024-5, was used as the reactant for the synthesis of 7-deacetyl-forskolin (7-DAFSK). Deacetylation of forskolin was carried out via classical carbonate saponification.

Deacetylation was carried out according to the reaction scheme shown in FIG. 1. First, 200 mg of forskolin (synthetic forskolin, Mercachem, batch MAMA07-024-5) and 200 mg of calcined Na₂CO₃ were stirred in 10 mL of methanol in a 250 mL round bottom flask, for 4 hours at room temperature. The reaction mixture was then allowed to stand overnight (20 hours).

The methanol was then distilled off (Rotavapor) and dissolved with about 20 mL CH₂Cl₂ from the flask. This was briefly sonicated (1 min). The 7-DAFSK formed goes into solution with CH₂Cl₂, precipitating the salts. The salts were filtered off (medium speed filters). Then the filtrate was washed with 2 n HCl in a separating funnel. The dichloromethane fraction was withdrawn and the solvent was distilled off in a rotavapor. The resulting white powder was analyzed by HPLC. A purity of over 99% was determined.

The structure of 7-DAFSK was verified by ¹H NMR ¹³C NMR. The measured ¹H and ¹³C NMR spectra of the obtained 7-DAFSK are identical to previously published spectra of this compound (J. Org. Chem. 2006, 71, 4619-4624). This confirmed the desired deacetylation of forskolin according to the reaction scheme shown in FIG. 1.

The obtained 7-DAFSK was further characterized by HPLC. Deacetyl forskolin and forskolin were eluted with a mobile phase of acetonitrile/water (55/45 v/v) at a flow rate of 0.6 mL/min (stationary phase: Nucleosil 120 3C18, 250×4 mm). A calibration curve was prepared for quantitative determination of the content of 7-DAFSK in complex with PVP. Calibration solutions with 7-DAFSK concentrations of 0.014; 0.028; 0.056 and 0.56 mg/mL were used for calibration.

### Example 2—Preparation of 7-DAFSK-PVP Complexes

Since 7-deacetyl-forskolin itself is only moderately soluble in water, 7-DAFSK was complexed with PVP to increase its water solubility. The 7-DAFSK complexes were prepared by the methods of the invention, hereinafter referred to as “heating methods”, and for comparison with conventional solution without heating (“dissolution methods”).

### 1. Preparation of 7-DAFSK-PVP Complexes by Heating PVP Above the Glass Transition Temperature (“Heating Process”)

7-DAFSK-PVP K12 Complex (Complex of 7-DAFSK with PVP K12)

For the complexation of 7-deacetyl-forskolin with PVP K12 (“Kollidon” 12 PF, Ph. Eur., USP, JP; BASF, average molar mass: about 2.5 kD), 20 mg of 7-DAFSK and 20 mg of PVP K12 were triturated in a porcelain crucible and 300 μL of ethanol was added and stirred. After 10 min, 100 μL of H₂O was added, stirred, and allowed to stand again for 10 min. The solution was then heated to 140° C. in a drying oven within 40 min and left in it for a total of 45 min. After cooling, 3 mL of H₂O was added and stirring was performed for 20-30 min. Then, the suspension was taken up with a syringe and filtered through 0.45 μm filter (hydrophilic). The crucible was rewashed with 3 mL H₂O and the wash solution was also filtered through the filter. The content of 7-DAFSK dissolved in the 6 mL H₂O was determined by HPLC. For the determination, the 7-DAFSK-PVP complex solution was diluted 1:10 with running medium. A mass fraction of 7-DAFSK in the total complex of 35 wt % was determined.

7-DAFSK-PVP K25 Complex (Complex of 7-DAFSK with PVP K25)

For the complexation of 7-deacetyl-forskolin with PVP K25 (FLUKA 81399, average molar mass: about 24 kD), 20 mg of 7-DAFSK and 20 mg of PVP K25 were triturated in a porcelain crucible and 300 μL of ethanol was added and stirred. After 10 min, 100 μL of H₂O was added, stirred, and allowed to stand again for 10 min. The solution was then heated to 185° C. in a drying oven within 40 min and left in it for a total of 45 min. After cooling, 3 mL of H₂O was added and stirred for 20-30 min. Then, the suspension was taken up with a syringe and filtered through 0.45 μm filter (hydrophilic). The crucible was rewashed with 3 mL H₂O and the wash solution was also filtered through the filter. The content of 7-DAFSK dissolved in the 6 mL H₂O was determined by HPLC. For the determination, the 7-DAFSK-PVP 25 complex solution was diluted 1:10 with running medium. A mass fraction of 7-DAFSK in the total complex of 17 wt % was determined.

### 2. Preparation of 7-DAFSK-PVP Complexes by Solution Process

7-DAFSK-PVP K12 Complex

20 mg of 7-DAFSK and 40 mg of PVP K12 were rubbed in a porcelain crucible and 300 μL of ethanol was added and stirred. After 10 min, 100 μL of H₂O was added, stirred, and allowed to stand again for 10 min. The solution was then allowed to stand at room temperature for 45 min, 3 mL of H₂O was added and stirred for 20-30 min. Then, the suspension was taken up with a syringe and filtered through 0.45 μm filter (hydrophilic). The crucible was rewashed with 3 mL H₂O and the wash solution was also filtered through the filter. The content of 7-DAFSK dissolved in the 6 mL H₂O was determined by HPLC. A mass fraction of 7-DAFSK in the total complex of 24 wt % was determined.

7-DAFSK-PVP K25 Complex

20 mg of 7-DAFSK and 40 mg of PVP 25 were rubbed in a porcelain crucible and 300 μL of ethanol was added and stirred. After 10 min, 100 μL of H₂O was added, stirred, and allowed to stand again for 10 min. The solution was then allowed to stand at room temperature for 45 min, 3 mL of H₂O was added, and stirred for 20-30 min. Then, the suspension was taken up with a syringe and filtered through 0.45 μm filter (hydrophilic). The crucible was rewashed with 3 mL H₂O and the wash solution was also filtered through the filter. The content of 7-DAFSK dissolved in the 6 mL H₂O was determined by HPLC. A mass fraction of 7-DAFSK in the total complex of 8 wt % was determined.

### Example 3—Determination of Water Solubility

For the solubility tests, the respective complex dried by means of rotary evaporator was used. A defined amount of water is added and then complex is added until an excess of 7-DAFSK-PVP is present. The supersaturated solution is sonicated, stirred and slightly heated (water bath 40° C.). The suspension is then filtered (0.45 μm filter) and the content of 7-DAFSK in the filtrate is determined by HPLC.

### Example 4—Characterization of the 7-DAFSK-PVP Complex

Mass Fraction of 7-DAFSK in the Total Complex

FIG. 2 shows a diagram of the determined content of 7-DAFSK in the 7-DAFSK-PVP K12 and 7-DAFSK-PVP 25 complexes, for which a ratio of 7-DAFSK to PVP of 20 to 20 was selected. A distinction is made here between the heating method and the dissolution method. For all complexes, clear differences can be seen in terms of the salts of 7-DAFSK between the two methods. Using the heating method, 8-9% higher salts of 7-DAFSK can be obtained in the final complexes. The 7-DAFSK-PVP K12 complex exhibits the highest content of 7-DAFSK (about 35 wt %).

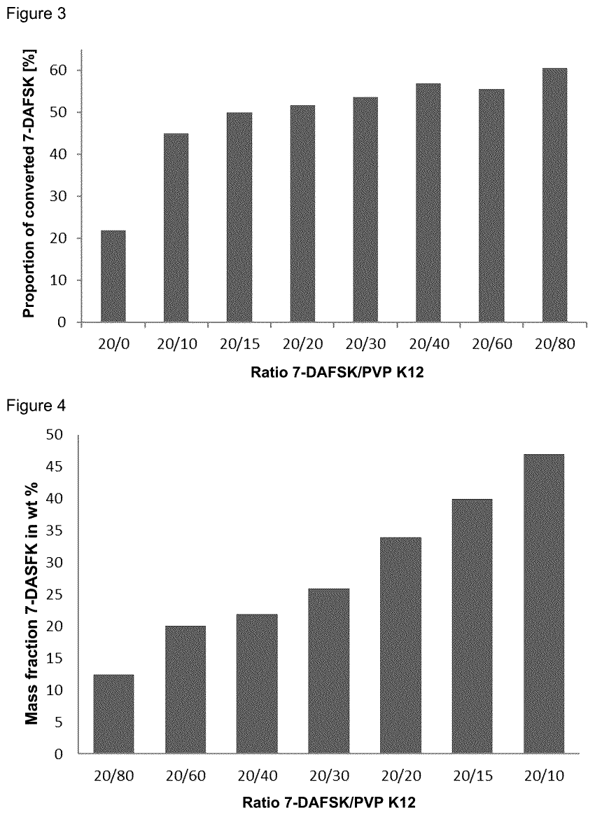

To optimize the content of 7-DAFSK in the total complex, the weight ratios of 7-DAFSK and PVP K12 used were varied (FIG. 3). Even by varying the ratios of 7-DAFSK/PVP K12, no more than 50-60% of the material used could be complexed with 7-DAFSK (20 mg) by this method, as can be seen from FIG. 3. It is clear that despite increasing the amount of PVP12, the final complexed amount of 7-DAFSK in the final complex does not change significantly. In order to find out how much non-complexed 7-DAFSK goes into solution during the dissolution process after preparation by heating, a control sample (without PVP12, only 7-DAFSK), which was treated in the same way as the complex samples, was examined. Here, less than a quarter of the 7-DAFSK used could be brought into solution. Complexation with PVP thus achieves an increase in 7-DAFSK solubility.

Even by increasing the amount of solvent before heating, it was not possible to bring larger amounts of 7-DAFSK into the complex with PVP K12.

FIG. 4 shows that even with very small amounts of PVP comparable results could be obtained.

The reported contents of 7-DAFSK in wt % were determined by HPLC. For the calculation it was assumed that the entire amount of PVP goes into solution when dissolved with the 6 mL H₂O.

Water Solubilities

Table 2 shows that a significant increase in the water solubility of 7-DAFSK is possible by complexation with PVP. The solubility of 7-DAFSK in complex with PVP is up to three times higher than the solubility of non-complexed 7-DAFSK. In contrast to 7-DAFSK, forskolin can hardly be dissolved with PVP due to its steric structure, nor can it complex with PVP.

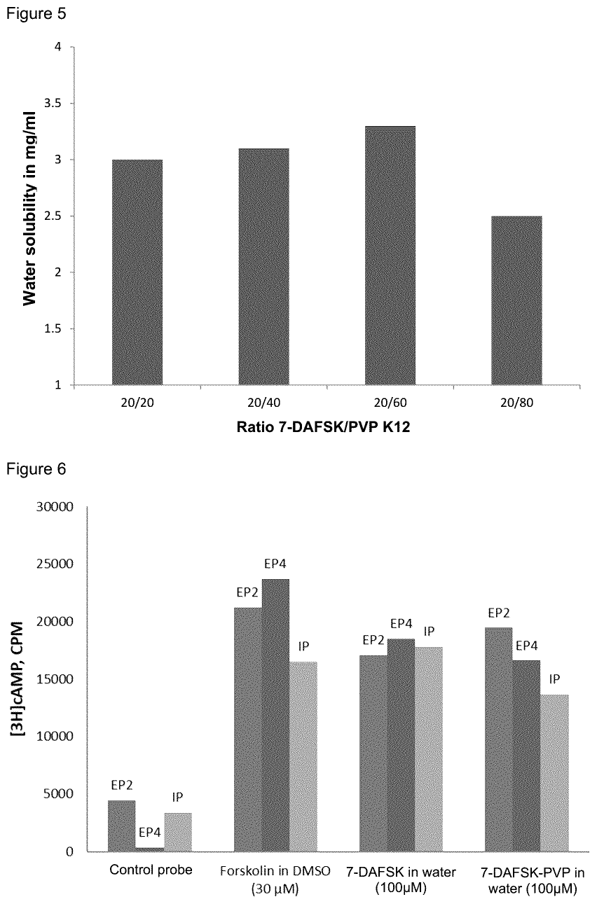

FIG. 5 shows that the water solubility does not change significantly with different ratios of DAFSK/PVP. The values vary between 2.5 mg/mL and 3.5 mg/mL. This is due to the fact that approximately the same proportion of 7-DAFSK (50-60%) of the complex is complexed at each ratio, which does not significantly change the water solubility of the complex.

TABLE 2
Water solubilities of different substances/complexes

| | PVP mass | |
|---|---|---|
| | fraction of | |
| Substance/complex | total complex | water solubility |
| FSK | — | 0.01 mg/mL |
| FSK-PVP12 | — | 0.001 mg/mL |
| | | (not detectable, |
| | | no detectable |
| | | complex |
| | | formation with |
| | | forskolin) |
| 7-DAFSK | — | 1 mg/mL |
| 7-DAFSK-PVP K12 | 35 wt % | 3 mg/mL |
| 7-DAFSK-PVP K25 | 17 wt % | 2 mg/mL |

### Example 5—Cell Culture Test: cAMP Assay

A cAMP assay was performed using HEK293 cells (human embryonic kidney cells, 1970) with the prostaglandin receptors EP2 or EP4, or the prostacyclin receptor IP. HEK293 cells were incubated with FCS and a radioligand ([³H] adenine). The formation of cAMP ([³H] cAMP) was monitored experimentally.

Activation of adenylycyclase (AC) by forskolin (dissolved in DMSO), 7-DAFSK, and 7-DAFSK-PVP, both dissolved in water, was compared. There are minor differences among the three forskolin derivatives in the activation of AC via EP2 and EP4 receptors, and no significant differences in the activation of the IP receptor (FIG. 6).

All three forskolin derivatives activate adenylyl cyclase via the receptors mentioned, even in the presence of treprostinil. Treprostinil alone (control sample) shows no activation of AC. The enhanced activity of forskolin, 7-DAFSK, and 7-DAFSK-PVP over the control is clearly evident in FIG. 6. Thus, these results indicate that 7-DAFSK-PVP is a suitable stimulator of adenylyl cyclase.

Assays were performed as follows:

Day 1: HEK293 cells stably expressing either EP2, EP4, or IP receptors were grown on a 6-well plate in 2 mL DMEM medium containing 10% FCS and 2 μCi [³H]-adenine and incubated for 16 hours.

Day 2: Cells were stimulated with 30 μM treprostinil alone or in combination with 30 μm forskolin in DMSO, 100 μM 7-DAFSK in water, or 100 μM 7-DAFSK in 7-DAFSK-PVP K12 complex in water (in fresh medium for 30 minutes at room temperature). Cells were then lysed with 2.5% perchloric acid (PCA) for 30 minutes on ice. The PCA extract was neutralized with KOH and [³H]cAMP was separated by sequential chromatography using DOWEX and alumina columns. Radioactivity was then measured using a scintillator.

An aliquot of PCA extract from each well was used to measure radioactivity.

### Example 6—Attempt to Prepare a Forskolin-PVP Complex by a Solution Method

20 mg FSK and 40 mg PVP 25 were triturated in a porcelain crucible and 300 μL ethanol (alternatively propanol) was added and stirred. After 10 min, 100 μL of H₂O was added, stirred, and allowed to stand again for 10 min. The solution was then allowed to stand at room temperature for 45 min, 3 mL of H₂O was added and stirred for 20-30 min. Then, the suspension was taken up with a syringe and filtered through 0.45 μm filter (hydrophilic). The crucible was rewashed with 3 mL H₂O and the wash solution was also filtered through the filter. The content of FSK dissolved in the 6 mL H₂O was determined by HPLC. A mass fraction of FSK in the total complex of <0.01 wt % was determined. FSK was not detectable in the filtrate by HPLC.

1. A composition comprising a complex of 7-deacetyl-forskolin (7-DAFSK) and polyvinylpyrrolidone (PVP).

2. The composition according to claim 1, characterized in that the PVP has an average molar mass relative to the weight average molar mass of from 1 to 40 kD.

3. The composition according to claim 1, characterized in that the 7-DAFSK is derived from synthetic forskolin.

4. The composition according to claim 1, characterized in that the average mass fraction of 7-DAFSK in the total complex is at least 50% by weight.

5. A process for the preparation of the complex of claim 1, characterized in that a mixture of 7-DAFSK and PVP is heated to a temperature which is above the glass transition temperature of the PVP.

6. The process according to claim 5, characterized in that a solvent is added to the mixture of 7-DAFSK and PVP.

7. The process according to claim 5, characterized in that the mixture is kept at a temperature above the glass transition temperature of the PVP for at least 5 minutes.

8. The process according to claim 5, comprising the steps of:
a) Preparing a mixture of 7-DAFSK and PVP and addition of a solvent, preferably an alcohol, preferably ethanol, to the mixture,
b) Adding water or an aqueous solution to the mixture,
c) Heating the mixture above the glass transition temperature of the PVP for at least 5 minutes,
d) Cooling of the mixture and dissolving in water or an aqueous solution, and
e) Removing undissolved components, optionally by filtering.

9. The composition according to claim 1, further comprising pharmaceutically acceptable ingredients.

10. (canceled)

11. The composition according to claim 9, characterized in that the composition contains 7-DAFSK in a concentration of at least 25 mg/L, at least 100 mg/mL, at least 250 mg/mL, at least 500 mg/mL, at least 1000 mg/mL, at least 2000 mg/mL, at least 3000 mg/mL or at least 4000 mg/mL.

12. The composition according to claim 9, wherein the composition is formulated for intravenous, oral or inhalation administration.

13. A method of treating cardiovascular disease, bronchial asthma, obesity, glaucoma, pulmonary disease, joint inflammation, or osteoarthritis/arthritis, comprising the step of administering a therapeutically effective amount of the composition of claim 1.

14. The composition according to claim 2, characterized in that the PVP has an average molar mass relative to the weight average molar mass of from 2 to 25 kD.

15. The composition according to claim 14, characterized in that the PVP has an average molar mass relative to the weight average molar mass of about 2.5 kD.

16. The composition according to claim 4, characterized in that the average mass fraction of 7-DAFSK in the total complex is in the range of from 1% to 50% by weight.

17. The composition according to claim 4, characterized in that the average mass fraction of 7-DAFSK in the total complex is in the range of from 1% to 30% by weight.

18. The process of claim 6, wherein the solvent is water, ethanol, methanol, propanol, acetone, ethyl methyl ketone, dichloromethane, ethyl acetate, or a mixture thereof.
